(19) United States
(12) Patent Application Publication
(10) Pub. No.: US 2021/0077800 A1
(43) Pub. Date: Mar. 18, 2021

(54) A SYSTEM AND A METHOD FOR TREATING TUMORS, ESPECIALLY INTRACRANIAL TUMORS

(71) Applicant: CARDIO TECHNOLOGY SP. Z O.O., Lublin (PL)

(72) Inventors: Marta MICHALCZYK, Karczew (PL); Kamil ADAMCZYK, Lublin (PL); Janusz Stanislaw FRACZEK, Warszawa (PL)

(21) Appl. No.: 16/612,136

(22) PCT Filed: May 8, 2018
(86) PCT No.: PCT/IB2018/053210

§ 371 Date: Nov. 8, 2019

(30) Foreign Application Priority Data
May 8, 2017 (PL) P.421532

(51) Int. Cl. A61M 37/00; A61B 17/3205; A61B 18/14

(52) U.S. Cl. CPC A61M 37/0092; A61B 17/3205; A61B 2018/00083; A61M 2037/0007; A61M 2205/8243; A61B 18/1402

(57) ABSTRACT

The present invention relates to a system for treating tumors comprising an extracorporeal part (1) and the intracorporeal part (2), wherein the extracorporeal part (1) comprises a primary TET module (6) constituting a primary resonant circuit and an external control and power module (9) connected to the latter one, wherein the intracorporeal part (2) comprising a subcutaneous module (10) connected to an implant (11); wherein the subcutaneous module (10) comprising the secondary TET module (13) constituting a secondary resonant circuit that receives energy from the primary TET module (6), and the implant comprising at least two implanted insulated electrodes (15) and at least one ultrasonic generator (16). Moreover, the present invention is the method for treating tumors which uses the system according to the invention.

The object of the invention is a system and a method for treating tumors, especially intracranial tumors, applied in the therapy of intracranial tumors following tumor resection, which is used to inhibit the growth of tumor cells in the brain area, as well as to enhance the efficiency of chemotherapy by its greater penetration to the brain tissue. Moreover, the said system and method for treating tumors may be used to treat other types of cancers within the chest, abdomen and pelvis, e.g. lung cancer, pancreatic cancer, and ovarian cancer.

The International Agency for Research on Cancer estimated that in 2012 around 12.7 million people were diagnosed with cancer worldwide, whereas nearly 7.5 million died as a result of a cancer-related illness. In Europe, around 1.7 million individuals are diagnosed with cancer each year, where for half of them the illness is fatal. In Poland, around 150,000 new cases and 90,000 deaths are reported each year. Thus, the statistics prove that cancer incidence poses a serious health and social problem.

Among cancer types, an intracranial neoplasm, also called a brain tumor, should be indicated. Such cancer is characterized by the development of mutated cancer cells within healthy brain tissue.

There are two main types of intracranial neoplasms: malignant and benign. Further, malignant tumors may be divided into primary tumors—which originate from and develop within the brain tissue, and secondary—also known as metastatic—tumors which had originally formed in another organ and subsequently metastasized to the brain.

Primary brain tumors are diagnosed in around 250,000 people every year worldwide, constituting around 2% of all cancers.

Over 40% of primary intracranial neoplasms are gliomas—one of the most malignant brain tumors, with the median survival rate of 5-15 years for patients with low malignancy tumors and 9-12 months for high malignancy tumors.

Although the clear causes of brain tumor development remain unknown, it has been proven that genetic predispositions increase its incidence. The risk of developing intracranial tumors is elevated in individuals suffering from: neurofibromatosis type I and II, Von Hippel-Lindau disease, tuberous sclerosis, retinoblastoma or Li-Fraumeni syndrome. Moreover, it has been proven that the transformation of a glial cell into a cancerous cell occurs through known gene mutations and amplification, i.e. presence of specific chromosomal losses—the so-called loss of heterozygosity.

Regardless of whether therapeutic methods are used, the majority of patients diagnosed with both benign and malignant brain tumors undergo surgical tumor resection—if such an operation is possible. Intracranial tumors characterized by diffuse, infiltrating, poorly separated from the healthy tissue or with low differentiation pose great difficulties for neurosurgeons during resection. Unfortunately, even full microscopic removal of highly malignant tumors does not guarantee full remission and often application of adjunct therapy in the form of chemotherapy or radiotherapy is necessary.

Treatment of brain tumors is a combined therapy. Depending on the needs and possibilities surgery, radiotherapy and/or chemotherapy treatments are applied. Very often, all the above-said forms of therapy are used or only some of them are combined. The therapy is adjusted to each patient individually taking into account their health condition and the degree of malignancy of the tumor.

As the neoplasm invades the nervous tissue, cancerous cells may begin to intertwine with regions of formerly unaffected and healthy tissue. If this occurs, the tumor is—if possible—resected with a surgical margin of healthy tissue. However, in the case of malignant tumors, despite macroscopic full resection, cancerous cells usually still remain in the postoperative tumor bed. Therefore, in the case of malignant gliomas, radiotherapy is often used as adjunct therapy. However, in the event of low malignancy tumors, radiotherapy or irradiation therapy are used in the case of incomplete tumor resection.

The fundamental radiotherapy technique to treat cancers of the central nervous system is fractional teleradiotherapy using a single daily dose of 1.8-2 Gy. The total dose depends on the histological type of cancer. In preparation for and planning of the radiotherapy treatment, it is necessary to construct individual thermoplastic masks with the points of radiation entry or field boundaries are marked.

Chemotherapy—the use of cytotoxic drugs—is another therapeutic method that is currently one of the basic methods of treatment for the majority of cancer types, including brain cancers. Cytotoxic drugs are anticancer medications which destroy cancerous cells by blocking their cell cycle and inducing genetically programmed mechanisms of cell death, namely apoptosis.

However, the application of chemotherapy is limited in the treatment of brain tumors as compared to cancers located in other organs, largely due to the high selectivity of the blood-brain barrier (a semipermeable barrier formed by endothelial brain cells connected by tight junctions), resulting in very limited penetration by most cytotoxic drugs. Consequently, the drug reaches the tumor by way of the process of diffusion. Due to the slow pace of the said process, in order to attain a high therapeutic drug concentration within the area of the cancer, it would be necessary to sustain a prolonged high concentration of the drug in the blood plasma which, in turn, would result in a very high systemic toxicity.

The barrier is formed by the aggregation of endothelial cells lining the apical membrane of cerebral blood vessels. Tight junctions in the apical membrane perform the key protective functions. These junctions are the main physical barrier for chemotherapeutics particle diffusing from the blood into the brain for therapeutic purposes.

Additionally, besides physical barriers, the blood-brain barrier is also composed of functional barriers—active efflux transporters that remove molecules which have penetrated the structural barriers, i.e. mainly tight junctions (TJs). Such transporters may be for example P-glycoproteins (present in the blood-brain barrier) that determine resistance to chemotherapy.

In the recent years, there have been efforts to implement various strategies to enhance chemotherapeutics delivery to brain tissue, including to brain tumors, including by the application of ultrasounds to reversibly disrupt the blood-brain barrier. Such approach involves intravascular administration of an agent (contrast) containing air bubbles (i.e. microbubbles). Once these bubbles enter the cerebral circulation they become exposed to ultrasounds and begin to vibrate which, in turn, leads to the temporary disruption of the blood-brain barrier. It does not, however, damage the membrane barrier. After some time, the barrier restores its initial functions.

Although several inventions exploiting this technology are already known on the market, each possesses its limitations—whether it is a matter of how effective the device is at disrupting the blood-brain barrier in the direct proximity of the tumor, or the issue of aseptic powering of the intracranial ultrasonic generator. Owing to the physical parameters of generated ultrasounds, such an intracranial ultrasonic generator is superior to extracorporeal generators.

One of the technologies currently available on the market is the invention described in patent application no. WO 2016097867 (A2) which acts upon the intracranial tumor through ultrasounds, the source of which is located on the meninges, under the skull, and is powered through a specialized needle which is temporarily inserted subcranially, into the energy receiving module.

Another invention protected by U.S. Pat. No. 8,977,361 (B2) which describes an invention that generates subcranial ultrasounds in order to disrupt the blood-brain barrier, and is powered by permanently installed wires running to the head surface connected to a power supply.

There have already been modern attempts at combining therapeutic electric field and ultrasounds in cancer therapy. Amongst the technologies available on the market, the invention filed under patent application no. WO 0240093 (A3) should be noted. This application described that an electric field is applied together with ultrasounds to create a synergistic effect leading to the ablation of either cancerous cells or entire tissues. Additionally, ultrasounds are used to sensitize the cells to an electric field and vice versa, i.e. the electric field is used to sensitize cells to ultrasounds. In the invention described in the patent application no. WO 0240093 (A2), the ultrasounds are administered directly to the tumorous tissue.

In conclusion, in this type of known systems combining two cellular impact technologies, the ultrasounds are applied to affect the blood-brain barrier, and not to the cancerous cells directly, to sensitize them to the destructive effect of the electric field.

The alternating electric field interferes with mitosis (the M phase of the cell cycle) in diving cells, however, has relatively little effect upon the non-cycling cells (remaining in G0 step). The healthy brain cells of an adult do not undergo frequent cell divisions, however in cancerous cells the divisions are much more frequent (but depend on the level of cancer malignancy). Therefore, the therapeutic action of the alternating electric field mainly blocks the cell cycle of cancerous cells.

Electric polarization and dielectrophoresis are essential physical mechanisms wherein alternating electric field affects the proliferation of cancerous cells.

The electric polarization is based on the fact that the majority of cellular structures and organelles are polar, and so they can also be considered polar dielectrics—composed of electrically asymmetrical molecules, i.e. they have a permanent dipole moment. However, regardless of whether the molecules possess a permanent dipole moment, after placing them in an external electric field, they acquire an additional—induced dipole moment, which forms from electron cloud deformation (electronic polarization) and atomic polarization, i.e. a shift in the atom or ion distribution in space under the influence of an electric field. Additionally, if the molecules of the dielectric are dipoles, and, at the same time, have the freedom of orientation, dipole polarization (the orientation of the molecules along the lines of the electric field) also has an effect onto the induced dipole moment. This is key during the metaphase step of mitosis, when mitotic spindles are formed from microtubules. Each spindle is formed with multiple fibers, so called microtubules. They are synthesized by tubulin subunit polymerization where each of them is a dimer of two very similar globular proteins: α-tubulin and β-tubulin bound by noncovalent bonds and possess large dipole moments.

When a cell is subjected to an alternating electric field, an electric field forms in its interior, which induces dipole moments of α-tubulin and β-tubulin leading to their alignment along the lines of the electric field. Consequently, instead of undergoing polymerization and merge with a microtubule, the tubulin components adapt to the external electric field, resulting in the inhibition of microtubule synthesis, disruption of metaphase, lengthening of mitosis and finally, cellular death.

Another phenomenon exploited in battling intracranial tumors is dielectrophoresis which, in one of the variant, induces the migration of cellular organelles along the alternating electric field. When the cell is not undergoing cellular division, i.e. remains in the G0 cell stage, like the majority of healthy brain cells, it is filled and surrounded by a continuous electric field and thus dielectrophoresis does not occur on its cellular organelles in its interior. However, if the cell within the electric field is undergoing metaphasis and begins cytokinesis, the affecting electric field ceases to be continuous. During cytokinesis, a contractile ring (composed of the proteins, i.e. myosin and actin) is formed along the equatorial plane. As the contractile ring tightens, the cleavage furrow develops where the electric field becomes denser than at the cellular poles. In consequence, the cellular organelles are attracted from sites of lower electric field density to sites of greater electric field density which triggers the structural disruption of the dividing cell. This interrupts mitosis, leading to cellular fragmentation and apoptosis. The majority of quickly proliferating cells in the brain are cancerous cells, which is why the above process inhibits cancerous growth with relatively little effect on healthy brain cells.

Generally, it is assumed that the speed at which substances penetrate brain tissues, is inversely proportionate to their molecule size and directly proportional to their lipophilicity. The function of the blood-brain barrier is to maintain homeostasis of the central nervous system and act as a barrier against blood-borne toxins. The blood-brain barrier is a dam that protects the brain from toxins. However, it has certain drawback. The most serious one is that it also blocks the flow of the overwhelming majority of drugs (around 98%), thereby inhibiting their entry into the brain. The exceptions are some lipid-related and lipophilic substances. Nevertheless, they do not find many therapeutic applications, as such drugs penetrate all cells in the body without serious side effects. It is worth mentioning the invention protected by the U.S. Pat. No. 7,917,227 (B2) wherein an alternating electric field is applied to treat intracranial brain tumors. The invention assumes the influence on brain tumors through the use of extracranial insulated electrodes. Nevertheless, such solution carries certain complications. Above all, the electric field generated in the central areas of the brain is not as strong and homogenous as the one generated in the proximity of the skull and it is more difficult to gain full control of the electric field distribution inside the skull. However, there is a possibility that a large tumor could be incompletely eliminated and subsequently, the remaining necrotic tissues that had been destroyed intracranially would provoke side effects and thus, the solution would be ineffective.

Following the surgical tumor resection or its destruction through radio- or chemotherapy, the so-called tumor bed is created. It is initially occupied with blood and air. Subsequently, the scarring processes take place and it fills with connective tissue. However, regardless of what remains inside the tumor bed, its density and hydration differs from the surrounding brain tissue and, consequently, the physical characteristics of electric field propagation in its interior also vary.

In consequence, by manipulating the alternating electric field onto the tumor bed within the brain, the electric field lines may be arranged in such a way that they will mostly run on the margins of the tumor bed, with a small extent of them directly penetrating it. This will occur when the contents of the tumor bed will have a greater permittivity than the surroundings. From a biological point of view, this is unfavorable as the brain tumor in the tumor bed may undergo regeneration and the cancer may recur.

It is worth bringing attention to the inventions disclosed in the following patent publications: U.S. Pat. Nos. 7,715,921 (B2), 7,706,890 (B2) and 7,565,205 (B2), in which the use of alternating electric field to treat intracranial brain tumors generated by electrodes located on the patient's scalp is described.

The invention protected by the U.S. Pat. No. 8,019,414 (B2) should also be drawn attention to as the technology simultaneously applies the therapeutic use of alternating electric field and chemotherapy. Furthermore, it is also worth referring to the invention filed under the U.S. Pat. No. 8,229,555 (B2) as it describes the use of an intracranial probe with insulated electrodes generating an alternating electric field, powered by wires permanently directed to the head surface and connected to a power supply. In the said invention, the necessity of wires powering an intracranial probe damages the skull and scalp barrier, which, in turn, results in an infection of the brain and meninges.

It is worth bringing attention to the TET technology (Transcutaneous Energy Transmission), i.e. intracorporeal energy transmission. The said technology was grounded on the invention filed under the U.S. Pat. No. 3,727,616 (A) that uncovered the principle of exploitation of electromagnetic energy transmission. The invention filed under the U.S. Pat. No. 5,350,412 (B1) should also be noted. It takes advantage of its wireless transmission method in the form of optical transmission with an intracorporeal device, which, in its standard version, does not interfere with the electromagnetic field generated by TET.

One of the latest solutions is offered by the invention filed under the U.S. Pat. No. 9,125,242 (B2), which regulates the power transmitted by the TET technology through regulating the frequency of the current flowing through the primary winding, i.e. extracorporeal-transmission and secondary intracorporeal-receipt. As the system reaches its resonant frequency (i.e. tuning in), the power is increased, whereas along with receding away from the resonant frequency (i.e. tuning out), the power is decreased.

The technical problem posed before the present invention is to propose such a system for treating intracranial tumors that will allow to penetrate the blood-brain barrier, and in a controlled manner, without damaging it, using natural blood vessel network in the brain will allow to administer intravascular chemotherapy which will penetrate to a greater degree into the brain tissues in the therapeutic drug concentration, without the need to generate very high, toxic systemic concentrations of these substances. Furthermore, it is desirable to provide a system that will be more effective than solutions known in the art and will provide greater patient safety when used. It is also important to ensure complete control of the electric field and/or ultrasound generation in the system for treating intracranial tumors, in particular including the intensity control (including amplitude) and the direction of the electric field and ultrasound impact, while ensuring their homogeneous distribution.

The present invention is a system for treating tumors comprising an extracorporeal part and intracorporeal part characterized in that the extracorporeal part comprises a primary TET module constituting a primary resonant circuit and an external control and power module connected to the latter one, wherein the intracorporeal part comprising a subcutaneous module connected to an implant, wherein the subcutaneous module comprising a secondary TET module constituting a secondary resonant circuit that receives energy from the primary TET module, and the implant comprising at least two implanted insulated electrodes and at least one ultrasonic generator.

Preferably, the extracorporeal part comprises at least two insulated electrodes for generating an alternating electric field.

Preferably, the subcutaneous module consists of at least two insulated electrodes for generating an alternating electric field.

Preferably, the subcutaneous module comprises a power supply path comprising at least the secondary TET module, the latter one comprising at least one secondary inductor of the secondary resonant circuit.

Preferably, the subcutaneous module further comprises a therapeutic path that comprises at least two subcutaneous insulated electrodes arranged on a PCB and a processor.

Preferably, the extracorporeal part further comprises an optical communication module, and the subcutaneous module comprises in the power supply path an optical receiver that cooperates with the optical communication module of the extracorporeal part.

Preferably, the extracorporeal part further comprises an optical communication module, and the optical receiver that cooperates with the optical communication module of the extracorporeal part is arranged in the power supply path in a subcutaneous power module.

Preferably, the primary TET module comprises a primary inductor, wherein the secondary TET module comprising at least one secondary inductor, the primary inductor and at least one secondary inductor parts constituting a transformer.

Preferably, the subcutaneous module is wired to the implant, preferably, through a secondary wire.

Preferably, the extracorporeal part further comprises an inverter and processor connected to: tha sound communication circuit, an optical communication circuit, an input interface, a wireless communication module connected to an antenna, a short-range wireless interface and at least one multiplexer.

Preferably, the at least one multiplexer is embedded in adhesive patches stuck on the head or is an integral part of the external control and power module.

Preferably, if the extracorporeal part holds at least two insulated electrodes, the latter ones are embedded in the adhesive patches stuck on the head and are powered through connecting wires connecting said at least two isolated electrodes (3) to the primary TET module from which the primary wire connecting the latter one to the external control and power module is running

Preferably, the subcutaneous module, further comprises in the therapeutic path a rectifying circuit and smoothing circuit connected to at least one secondary inductor and at least one multiplexer, wherein the processor controls generation of ultrasounds from at least one ultrasonic generator located in the implant, wherein, preferably, at least one secondary inductor, the optical receiver, the processor, the rectifying circuit, the smoothing circuit are located on the common PCB.

Preferably, the processor mutually communicates with the processor by means of light impulses emitted through the skin by the optical communication module that cooperates with the optical receiver by means of light impulses emitted through the skin.

Preferably, the at least one ultrasonic generator is in the form of a piezoelectric crystal.

Preferably, the external power source constitutes a set of non-reusable batteries, an accumulator, an ‘energy harvesting’ generator which produces electric power from heat, light or movements, and/or a computer.

Preferably, the intracorporeal part is made of sterile, biocompatible materials and/or materials that do not interfere with the MRI exam.

According to another aspect, the object of the invention is a method for treating tumors, especially intracranial tumors, characterized in that it comprises:
(a) a step of at least partial tumor resection;
(b) a step of locating the system according to any one of claims from 1 to 16 inside and on the body of a patient, wherein the implant being placed in the tumor bed left after a fragmentary or total tumor resection;
(c) a step of generation of ultrasounds and/or the step of an alternating electric field generation prior to a step of administration of chemotherapy; and
(d) the step of administration of chemotherapy .

Preferably, in the present invention, the distribution of the electric field during step (c) at any given time is resultant of the electric field of at least two pairs of electrodes that generate electric field in different directions, preferably, of the insulated extracorporeal electrode and the implantable insulated electrode or of the insulated extracorporeal electrode with the insulated electrode or of the implantable insulated electrode with the implantable insulated electrode or of the insulated intracorporeal electrode and the implantable insulated electrode or of the insulated intracorporeal electrode with the insulated intracorporeal electrode.

Preferably, in the step (c) an alternating electric field is generated, preferably in a continuous manner, of the frequency between 50 kHz and 500 kHz and/or the intensity of at least 0.5 V/cm, wherein the electric field being generated simultaneously in at least two directions.

Preferably, in the step (c) ultrasounds are generated, periodically or continuously, of the frequency of emission between 100 kHz and 10 MHz.

According to another aspect, the object of the invention is a use of the system according to any of claims 1-17 in treating tumors.

Preferably, the system is used in treating intracranial tumors.

More preferably, the system is used in treating intracranial tumors in combination with chemotherapy, wherein the system generates ultrasounds and/or alternating electric field prior to the administration of chemotherapy.

The system for treating intracranial tumors with the use of a combined technique of continuous alternating electric field and ultrasounds powered by transcutaneous energy transmission is more advanced invention than the solutions currently available on the market. Its fundamental asset is the possibility of generating a stronger electric field in the internal segments of the organism, especially the brain, and especially within the area of the tumor, i.e. the tumor bed, without the need of generating very high energies of electric field through epidermal electrodes.

The present system for treating tumors, especially intracranial tumors, in order to maximize its efficiency, assumes the use of insulated intracorporeal, especially intracranial electrodes (implant) to amplify the electric field inside the patient's body, especially inside the brain, particularly in the brain stem area and further the use of ultrasounds to disrupt the blood-brain barrier and block the pumps (e.g. P-gp) active in the blood-brain barrier within the tumor bed and surrounding tissues.

The present system for treating tumors, especially intracranial tumors, therapy is more effective than the existing modern solutions because the ultrasonic generator is placed within the tumor bed and thus the acoustic pressure of ultrasounds in this zone is higher in the present invention, the blood-brain barrier is disrupted to a greater extent, whereas the chemotherapeutic penetrates the tumor and other tissue, especially the brain tissue, more effectively. Additionally, in the present invention, in the case of use for intracranial tumor therapy, the energy needed to power the implant is transmitted without destruction of the skull and scalp, and hence, the risk of infection to the brain and surrounding meninges is significantly lowered.

Additionally, it should be noted that if the system is used as per the invention for intracranial tumor therapy, the ultrasonic generator (implant) is located not directly on the surface of the meninx under the skull, but within the tumor bed. As a result, the ultrasounds are generated in multiple planes nearby the tumor bed, which results in a much greater disruption of the blood-brain barrier within proximity to the tumor bed. Additionally, the implant may also deliver therapy in the form of alternating electric field to destroy cancerous cells.

Moreover, the implantable electrodes used in the system intensify the electric field within the tumor bed and additionaly generate a multidirectional alternating electric field on the margins of the tumor bed, said field actingduring the same time unit in different directions throughout the entire duration of the treatment. This is particularly relevant for destructing cancerous cells remaining in the tissue surrounding the tumor bed after the resection.

The mechanism of action of alternating electric field therapy is similar to the mechanism of the chemotherapy, which is the inhibition of the cell cycle and induction of genetically programmed mechanisms of cell death in cancer cells.

An unquestionable advantage of the system is the fact that the device redistributes the electric field inside the organism, especially inside the skull, in a more continuous manner than any devices available on the market, due to the use of the intracranial implant with electrodes. If the system according to the invention is used to treat intracranial tumors, an electric field generated infratentorially (including within the brain stem—an enormously vital part of the brain) is much stronger than an electric field generated exclusively by epidermal electrodes and, most importantly, has a therapeutic application. On the other hand, due to the use of the intracranial module (implant), it is easier to modify the electric field distribution in such a way that would increase its intensity in the areas of cancerous growth and, at the same time, weaken its intensity in areas where electric field stimulation is not recommended or areas that do not show signs of neoplasms.

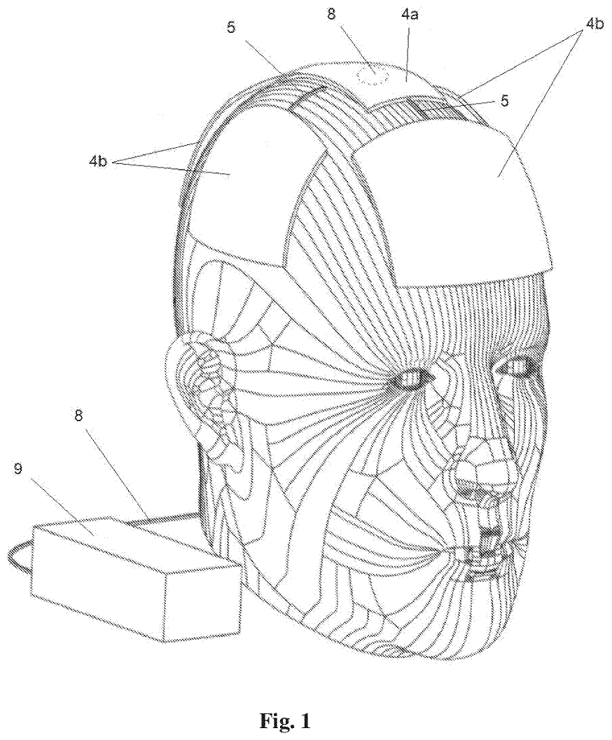

The object of the invention was exemplified on the drawing in which FIG. 1 presents a schematic illustration of the system for treating tumors according to one embodiment of the invention, especially adapted to the treatment of intracranial tumors, placed on a patient's head, seen from the outside;

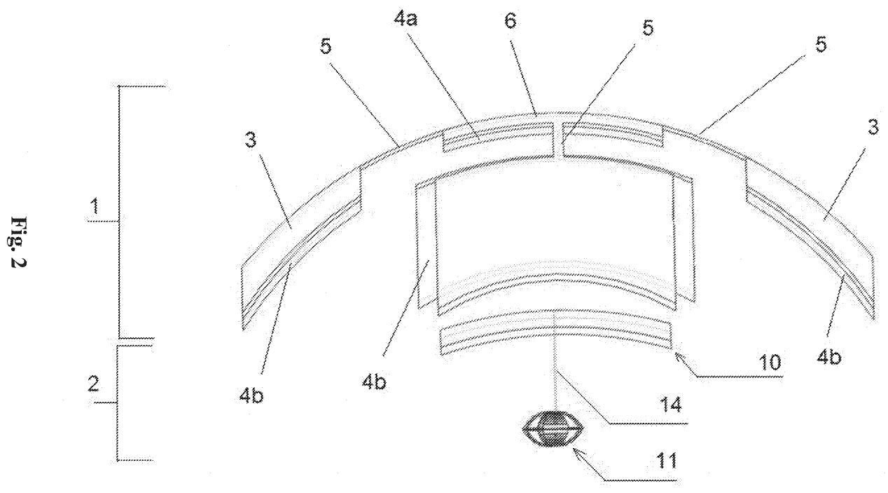

FIG. 2 shows a schematic illustration of the system for treating tumors, especially adapted to treat intracranial tumors with visible extracorporeal and intracorporeal parts;

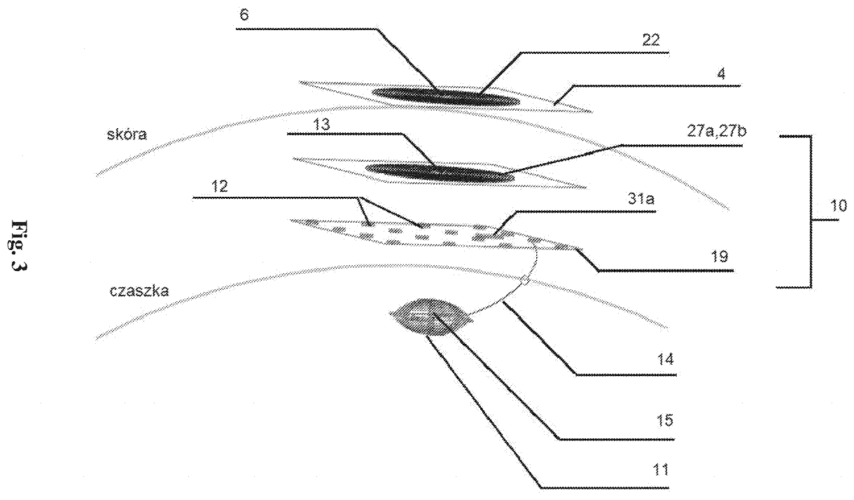

FIG. 3 shows a scheme similar to FIG. 2 that represents the structure of the extracorporeal part;

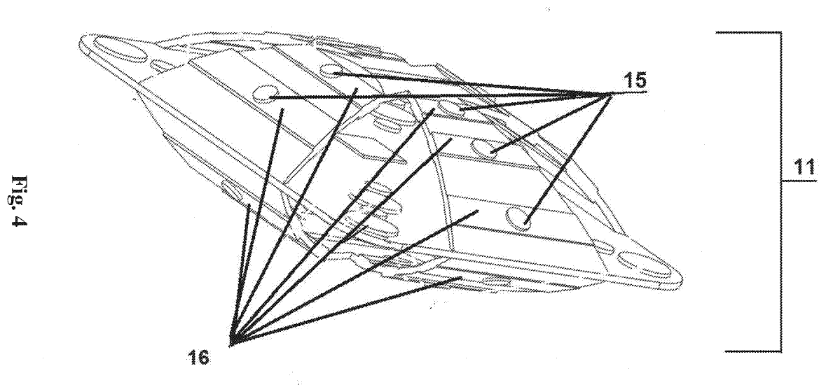

FIG. 4 shows an axonometric view of the intracorporeal implant;

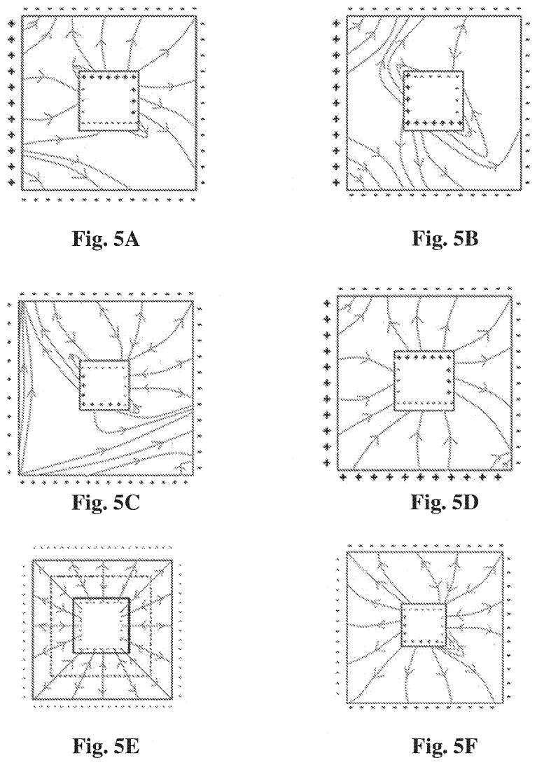

FIG. 5A-F show examples of electric field distributions in the system for treating tumors according to the present invention, in particular adapted to treat intracranial tumors;

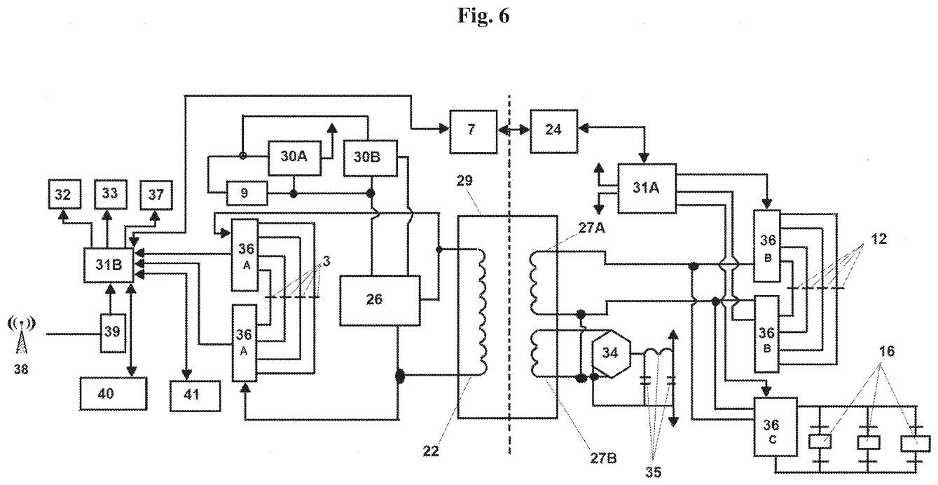

FIG. 6 shows a block diagram of the system for treating tumors according to the present invention, especially adapted to treat intracranial tumors, whereas

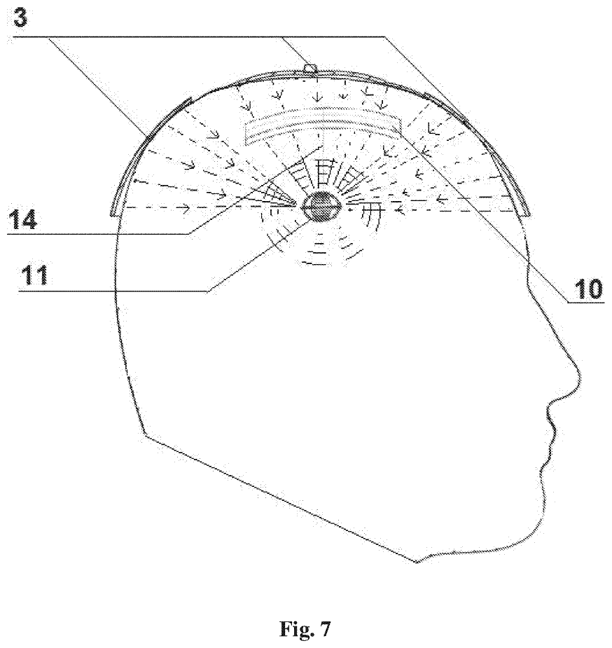

FIG. 7 illustrates propagation of electric field and ultrasounds when using the system for treating tumors, when in the intracorporeal implant, located especially intracranially, all electrodes have a negative polarity.

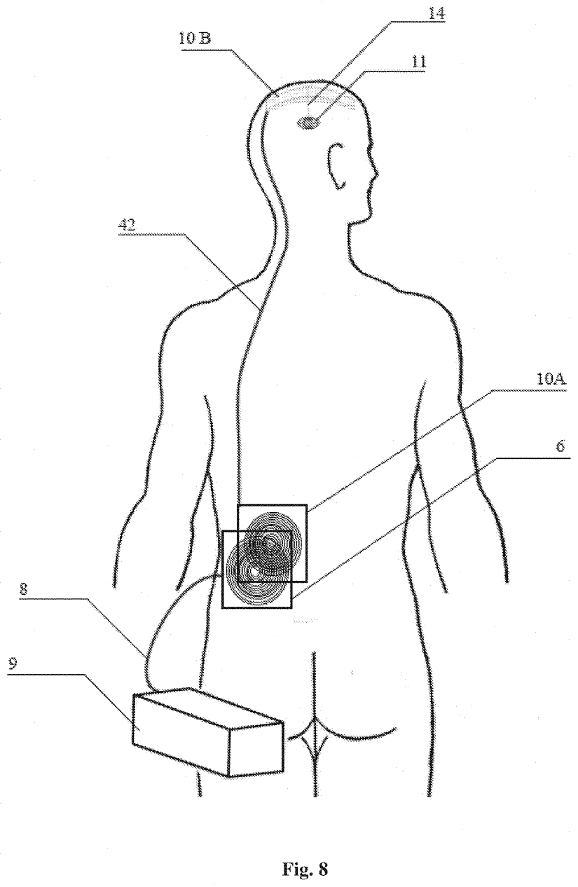

FIG. 8 shows a schematic illustration of the system for treating tumors according to another embodiment of the invention, especially adapted to treat intracranial tumors, placed on the lumbar part of the patient, seen from the outside.

### DESCRIPTION OF EMBODIMENTS

The system for intracranial tumor therapy according to one embodiment of the present invention is shown in FIGS. 1-4 and on the block diagram in FIG. 6.

The system for treating tumors according to the invention, especially for treating intracranial tumors, is composed of two parts: the extracorporeal part 1 and the intracorporeal part 2. The intracorporeal part comprises the subcutaneous module 10 and the implant 11. The intracorporeal part 2 is made of sterile and biocompatible materials allowing exposure to tissues of the subcutaneous implant as well as of the implant 11 over the course of several months and preferably materials that would not interfere with the MRI exam.

The extracorporeal part 1 is composed of components which are placed on a patient's body, especially on the head as in one embodiment, and of components which constitute an external equipment. In one of embodiments as shown in FIG. 1 the components placed on a patient's body in the proximity of the treated cancer constitute a central adhesive patch 4a which comprises the primary TET module 6 (as shown in FIG. 3) and at least two adhesive patches 4b with insulated extracorporeal electrodes 3 to generate alternating electric field. Preferably, the system comprises five adhesive patches 4b with extracorporeal electrodes 3 for generating alternating electric field. However, it should be noted that the presence of the extracorporeal electrodes 3 is only preferable, and their location depends on the type of cancer which is treated.

In one variant additionally built-in multiplexers 36A can be arranged in adhesive patches 4b with extracorporeal electrodes 3. In an alternative embodiment of the device, the multiplexers 36A can also be located in the external control and power module 9.

The primary TET module 6 in the central adhesive patch 4a is the primary resonant circuit and, as sown in FIG. 2 and FIG. 3, it is located directly over the intracorporeal part, especially over the subcutaneous module 10. In one variant of the invention, in the central portion of the primary TET module 6 there is an optical communication module 7 with the INV1 converter 30A and the INV2 converter 30B (as shown in FIG. 6), as well as the primary inductor 22. The primary inductor 22 also forms part of the transformer 29 (as schematically shown in FIG. 6). In an alternative embodiment of the present device, the INV1 converter 30A and INV2 converter 30B can also be located in the external control and power module 9.

The extracorporeal part 1, that is not placed on a patient's body and that constitutes the external equipment, is composed of the abovementioned primary wire 8 connecting the components placed on a patient's body with the external source of energy located in the external control and power module 9. Therefore, as shown in FIG. 1 and FIG. 2, the extracorporeal part 1 comprises two types of wires. The first types are the connecting wires 5 that connect the adhesive patches with insulated electrodes 3 with the TET module 6. The latter type is the primary wire 8 connecting the external control and power module to the primary TET module 6 which then transmits energy to other insulated electrodes 3 through connecting wires 5 also connected to the primary TET module 6. Additionally, the primary wire 8 is an optical fiber which assures the connection of the said optical communication module 7 located in the primary TET module 6. The primary wire 8 assures communication and power supply between the primary TET module 6 located in the central adhesive patch 4a, as well as the electric components located in the other adhesive patches 4b, and the external control and power module 9, which a patient carries in a customized case attached to a belt or in the backpack.

The external control and power module 9 may be a computer, laptop or other electronic module. As sown in FIG. 6, the external control and power module 9 consists of an OUT processor 31B, a sound communication circuit 32 (e.g. speaker), an inverter 26, a display 33 or set of LED lights 33, a button or set of buttons 37 (e.g. keyboard), an antenna 38, a wireless communication module 39, a wired interface 40 and a short-range wireless interface 41. A specialist in the art will know that the exemplified location of the abovementioned modules may vary within the extracorporeal part 1, which means that the extracorporeal part 1 may be composed of a greater number of separate electronic modules than was indicated. In particular, the components indicated as the parts of the control and power module 9 may be dispersed throughout several separate electronic modules.

The OUT processor 31B located in the external control and power module 9 controls the extracorporeal part 1. It communicates with the user through the sound communication circuit 32, display 33 or set of LEDs 33 (or the display) and buttons 37. The OUT processor 31B can communicate with other devices through a wireless interface, e.g. Bluetooth, Wi-Fi, or by wired connections through wired interface, e.g. USB or Ethernet.

As shown in FIG. 2 and FIG. 3, the main element of the system is the intracorporeal part 2, further comprising the subcutaneous module 10 and the implant 11, which is then placed inside a patient's body during the neurosurgery to remove the tumor, especially the brain tumor. The implant 11 is positioned in the bed of the tumor, especially the brain tumor, and can then directly affect the brain tissues and cancerous cells. Its therapeutic effect is not impeded by natural barriers such as skin tissue, muscle or skull. The subcutaneous module 10 is positioned under the skin also during the neurosurgical tumor resection, which is described in detail below.

As shown in FIG. 2 and FIG. 3, the intracorporeal part 2 holds an intracranial module 11 (implant). The implant 11 consists of implantable insulated electrodes 15 to generate alternating electric field and an ultrasonic generator 16. The implant (11) is positioned in the bed of the tumor, especially the brain tumor, after surgically removing at least a part of the cancerous growth. The secondary wire 14 runs from the implant 11 through an opening in the skull and connects to the subcutaneous module 10 from which it is powered.

As shown in detail in FIG. 3, the intracorporeal part 2 further comprises the subcutaneous module 10 containing subcutaneous insulated electrodes 12 that generate alternating electric field. However, it should be noted that their presence is only advantageous. They can perform a supplementary or substitute role with respect to the extracorporeal insulated electrodes 3. Additionally, the subcutaneous module 10 holds the secondary TET module 13 that received energy from the primary TET module 6 from the extracorporeal part 1. The secondary TET module powers all other parts of the subcutaneous module 10 and the implant 11. In terms of the mechanical structure of the subcutaneous module 10, as shown in FIG. 3, the subcutaneous module holds the PBC 19 which further comprises: a circuit of secondary inductors 27A, 27B which form the secondary TET module 13, an optical receiver 24, an IN processor 31A, a rectifier circuit 34, a smoothing circuit 35, and a component of the transformer 29. As mentioned above, the intracorporeal part 2 of the system according to the invention also comprises the secondary wire 14 that connects the subcutaneous module 10 with the intracranial module 11 (implant) to enable power supply to the implant 11.

As mentioned above, the main function of the extracorporeal part 1 is to power the intracorporeal part 2 because the intracorporeal part 2 does not possess its intrinsic source of power due to two reasons. First of all, connecting the intracorporeal part 2 of the system, especially the implant 11, with an extracorporeal, external power source 9 through the secondary wire 14 would bring about a huge risk of brain and meninges infection, even if it were to occur for a short period of time. Additionally, the use of an internal power source in the form of e.g. an implantable battery would greatly restrict the electrical parameters of the device and subject the patient to additional stress connected to surgeries involved with the change of batteries and re-implantation of the device. That is why, the intracorporeal part 2 in the present system does not possess an intrinsic power source nor does it in any way store the externally supplied energy. In turn, it immediately utilizes the energy received by means of the TET technology in cooperation with the extracorporeal part 1.

In terms of the power supply to the intracorporeal part 2, the energy transmission from the extracorporeal part 1 occurs between the epidermal primary resonant circuit of the primary TET module 6 comprising the primary inductor 22, and the secondary TET module 13 containing the secondary inductors 27A and 27B located in the upper component of the subcutaneous module 10.

As shown in detail on the electrical schematic diagram of FIG. 6, the system of wireless transcutaneous energy transmission comprises two transmission paths (energy and data) and components responsible for recording measurements and for its control. The energy transmission path comprises: a source of direct current, i.e. an external power source in the external control and power module 9, an inverter 26, a primary inductor 22 and a magnetically coupled circuit 27 of secondary inductors 27A, 27B.

As mentioned above, the inverter 26 is one of the components of the electrical energy transmission system located in the primary portion of the external control and power module 9. The inverter 26 transforms the powering continuous current into an AC or alternating current, thus allowing energy transmission through the primary inductor 22 and a circuit of magnetically coupled secondary inductors 27A, 27B.

In the present state of technology, there is an abundant variety of inverters, which could be used to achieve energy transmission. For example, there are class D and class DE resonant full bridge and half bridge inverters, class E and class F inverters. There are also non-resonant inverters, e.g. controlled by a microprocessor.

In the present system, the transmitted power (energy) can be regulated in two ways. In the first case, by regulating the amplitude of the electric current flowing through a transmitting inductor (primary inductor 22), i.e. extracorporeal primary winding-proportional change in the amplitude of the AC current flowing through the transmitting inductor will result in a proportional change in the amplitude of the current which is being induced and is flowing through the receiving inductor (secondary inductor 27A, 27B), i.e. intracorporeal secondary winding. In the second case, by modulating the width of the pulse wave supplying the transmitting inductor. The change in the modulation of width of the pulse wave—Pulse Width Modulation (PWM)—results in the change of power transmitted into the receiving inductor. As the trajectory is brought in approximation with the symmetrical square wave trajectory, the power of transmission is increased.

The data transmission path creates an optical, transcutaneous, duplex data transmission circuit. However, data transmission occurs between the epidermal transmitter 23, which is a component of the optical communication module 7 located in the extracorporeal part 1, and between the subcutaneous optical receiver 24, which is a component of the subcutaneous module 10. It should be emphasized, that both the optical communication module 7 as well as the optical receiver 24 can act both as transmitters and receivers (of energy and data).

The extracorporeal part 1 is powered by an external energy source located in the external control and power module 9. The external power source can consist of a set of non-reusable batteries, an accumulator, an ‘energy harvesting’ generator which produces electric power from heat, light or movements, and/or a computer.

As shown on the schematic diagram in FIG. 6, the external power source 9 located in the external control and power module 9 powers the INV1 converter 30A which then generates voltage to the following systems of the control and power module 9: sound communication circuit 32, display 33, OUT processor 31B, wireless communication module 39, wired interface 40, short-range wireless interface 41.

The INV1 converter 30A also generates voltage that powers the systems in the components placed on a patient's body, i.e. the primary TET module 6, including the optical communication module 7, possibly the control element of the inverter 26, possibly extracorporeal multiplexers 36A and powers the INV2 converter 30B that powers the inverter 26 and possibly the extracorporeal multiplexers 36B.

The inverter 26 transforms the direct current into an alternating current, where the primary inductor 22 transmits energy to the circuit of secondary L1, L2 inductors 27A, 27B. The L1 inductor 27A powers the electrodes 12 to generate alternating electric field in the subcutaneous module 10, as well as the implantable electrodes 15 and ultrasonic generators 16 located in the implant 11. The L2 inductor 27B powers the rectifying circuit 34 and smoothing circuit 35—and in effect it powers the IN processor 31A and the optical receiver 24 located in the primary TET module and multiplexers 36.

As shown in FIG. 6, the circuit of secondary inductors 27A, 27B located on the PCB 19 and the primary inductor 22 (a part of the primary TET module 6) create the transformer 29. The circuit of secondary inductors 27A, 27B and the primary inductor 22 are coupled in such a way that the primary inductor 22 constitutes the primary winding of the transformer 29, whereas the L1 inductor 27A and L2 inductor 27B constitute the secondary winding.

The inverter 26 powers the primary inductor 22 and insulated electrodes 3 through an alternating current. The OUT processor 31B, through the multiplexers 36 MUX1 and MUX2, determines which of the exit poles of the inverter 26 are to be connected to the insulated electrodes 3. This will directly influence whether or not they whether they will interfere with the electric current in regard to the electric current on given insulated electrodes 3 and on the implantable insulated electrodes 15.

The IN central processing unit (CPU) 31A which is located on the PCB 19 and which controls the intracorporeal part mutually communicates with the OUT processor 31B which controls the extracorporeal part through the optical communication module 7, which comprises an optical receiver 24 and an extracorporeal optical communication module 7. The optical communication module 7 and the optical receiver 24 communicate through light impulses (e.g. infrared light) emitted transcutaneously, which assures communication between the extracorporeal part 1 and intracorporeal part 2 that does not require rupturing the skin.

The intracorporeal part 2 is powered through one or more inductors of the secondary winding. The L2 inductor 27B powers the IN processor 31A. The current powering the IN processor 31A is rectified by the rectifying circuit 34 and the smoothing circuit 35. The L1 inductor 27A powers the subcutaneous insulated electrodes 12, implantable electrodes 15 and ultrasonic generators 16.

By the use of multiplexers 36, the IN processor 31A determines the pole of the L1 inductor 27A to which the subcutaneous insulated electrodes 12 and implantable electrodes 15 will be connected, which will directly influence whether or not they will interfere with the electric current in regard to the electric current on the given extracorporeal insulated electrodes 3 and with the electric currents between each other. The IN processor 31A uses the multiplexer 36 to determine whether to activate or de-activate the ultrasonic generators 16.

It is also possible to introduce a third receiving inductor (secondary winding) which would power exclusively the ultrasonic generators 16. In such a circuit, the secondary L1 inductor 27A would only power the subcutaneous insulated electrodes 12 and the implantable electrodes 15, the L2 inductor 27B would only power the IN processor 31A and possibly the intracorporeal multiplexers 36 as well as the optical receiver 24, whereas the third inductor would power exclusively the ultrasonic generators 16. It is also possible that the said third inductor would directly power only the electronic system controlling and powering the ultrasonic generators 16 and which would generate suitable course on the ultrasonic generators 16.

Generally, as shown in FIG. 6, a part of the components contained in the subcutaneous module 10 is the power supply path 10A i.a. to power and control the implant 11, and the other part creates the therapeutic path 10B to generate alternating electric field The power supply path 10A consists of the secondary TET module 13 that holds the L1 inductor 27A and L2 inductor 27B of the secondary resonant circuit. On the other hand, the therapeutic path 10B is composed of subcutaneous insulated electrodes 12 placed on the PCB 19 and the control system as shown in FIG. 6, including the processor 31a.

In the first embodiment of the system according to the invention, both the power supply path 10A and the therapeutic path 10B are located in the subcutaneous module 10, on a single PCB 19.

As shown in FIG. 8, in another embodiment, it is, however, possible that to located power supply path 10A in other place than the therapeutic path 10B (in such case each path holds its PCB and microprocessor) Thus, it is possible to predict that the power supply path 10A will be located in a separated subcutaneous power module 44 also located intracorporeally, but outside the subcutaneous module 10 on the patient's hear. The subcutaneous power module 44 can preferably be located in the lumbar region of the spine and is placed between the dorsal skin and the back muscles. The components of the extracorporeal part 1 are also positioned in that lumbar region extracorporeally.

If the components of the power supply path 10A and the therapeutic path 10B are separated, then these two modules are connected to an inter-module wire 42 (as shown in FIG. 8) that runs subcutaneously as a whole. Additionally, the primary TET module 6, which comprises a complementary primary resonant circuit with an inductor 22, is always placed over the power supply path 10A containing the secondary TET module 13, thus the primary TET module containing the complementary primary resonant circuit with an inductor can also be placed in other place than the head.

As mentioned above, selected components of the extracorporeal part 1 are placed on a shaved head of a patient, whereas the intracorporeal part 2 is installed by a neurosurgeon inside the patient's body. In particular, the implant 11 is placed within the intracranial space, whereas the subcutaneous module 10 is placed on the surface of the skull under the scalp.

The intracranial module 11 is implanted during the surgical resection of the primary brain tumor, which is described in detail below.

The following pre-requisites must be met for the system to operate properly. First of all, the patient in whom the system for treating tumors according to the invention is to be implanted, should be adequately qualified, i.e. must be diagnosed with a malignant cancer, e.g. intracranial neoplasm and the tumor should be located in an area that would allow for (at least partial) surgical resection. Moreover, the patient must be in a sufficiently good physical condition which would permit undergoing surgical treatment, i.e. neurosurgery. If the first condition is satisfied, the patient is qualified for the treatment according to the invention and the intracorporeal part 2 is placed in the patient's body in the following manner.

If the system according to the invention is applied to treat intracranial tumor, firstly, the neurosurgeon performs a craniotomy—an operation during which brain tissues are surgically removed and the skull is open. Next, the meninges are dissected allowing the neurosurgeon to access the tumor tissue. Then, the neurosurgeon removes the tumor, as thoroughly as possible, and assures the hemostasis of surrounding tissues. Following the tumor resection, the tumor bed is formed, i.e. the area formerly occupied by the cancerous growth.

Afterwards, the neurosurgeon proceeds to the surgical implantation of the intracorporeal component 2 of the device according to the invention which is comprised of the implant 11, the subcutaneous module 10 and secondary conductor 14 which connects the implant 11 to the subcutaneous module 10 placed under the skin, but on the surface of the skull.

Firstly, the implant 11 is placed in the tumor bed, formed after the tumor resection. The implant 11 is then connected to the subcutaneous module 10, which is then placed inside the patient's body, through the secondary conductor 14. After assuring that the intracranial module 11 is positioned correctly inside the tumor bed, i.e. it is whole inside the tumor bed, and that the secondary conductor 14 does not run over the upper portion of the implant 11 and that there is no hemorrhage from the tumor bed, the neurosurgeon can proceed to close up the meninges. Next, the neurosurgeon proceeds to drill an opening on the side of the fragment of the skull which had formerly been removed during the craniotomy, in such a way that the opening is confined from three sides by the edges of the removed skull, and open from one side. The secondary conductor 14 is placed through the said opening, and subsequently the said fragment of the skull with the secondary conductor 14 is re-installed in the skull to cover the tumor bed containing the placed implant 11. During the procedure, the implant 11, the secondary conductor 14 and the subcutaneous module 10 are connected and located in a sterile environment.

Subsequently, the neurosurgeon fixes the removed skull fragment into the skull by non-absorbable cords placed in the previously drilled openings both on the edges of the removed skull fragment, and the remaining part of the skull itself. Additionally, the neurosurgeon drills two openings onto the edges of both edges of the removed skull fragment and the skull, through which she/he threads non-absorbable cords, however, does not tie them together after placing back the skull fragment, but leaves them loose. The result of such procedure is restoring and re-stabilizing the lobe of the skull and leaving the 8 loose cords threaded through the openings in the ablated lobe of the skull and in the skull itself through which the secondary wire 14 runs thereby connecting the implant 11 to the subcutaneous module 10 which still has not been placed, which will be installed by the neurosurgeon as the next step.

For that purpose, the neurosurgeon assesses the excess of the secondary wire 14, if any, protruding from the opening and, after adapting the subcutaneous module 10 to the size of the skull, (s)he rolls up the secondary wire 14 and arranges it on the surface of the skull. Next, the neurosurgeon threads the loose cords which have already been threaded into the openings in the skull and the openings on the edges of the subcutaneous module 10, thereby fixing the subcutaneous module 10 to the skull in such a way that the lower portion of the subcutaneous module 10, so called therapeutic path 10B, containing the subcutaneous insulated electrodes 12, meets with the surface of the skull. However, the upper portion of the subcutaneous module 10, so called the power supply path 10A, contains the L1 inductor 27A and L2 inductor 27B of the secondary resonant circuit. Next, the neurosurgeon springs the cords and ties knots on them in such a way that they remain tightened and are able to hold the subcutaneous module 10 motionless. Then, the neurosurgeon stitches the muscles and fasciae removed during the surgery, so that they directly cover the upper portion of the subcutaneous module 10 which holds the subcutaneous resonant circuit that consists of inductors 27A and 27B that also constitute the secondary transformer circuit, i.e. the secondary TET module 13. The secondary transformer circuit 29 will be located under the surface of the skin on which the primary TET module containing the complementary primary resonant circuit with the inductor 6 will be placed. After stitching the muscles and fasciae as well as the subcutaneous tissue and skin, the skin is disinfected, wound dressing is applied, the surgery of brain tumor removal and intracorporeal part 11 implantation is concluded.

Subsequently, the patient is woken up from general anesthesia, moved to the post-operative ward and sent home if no further complications occur. After several weeks, when the post-op wound has healed, the patient is ready to have the extracorporeal part 1 installed and can begin the proper cancer therapy.

Correct fitting of the extracorporeal part 1 involves the following: proper placement of the adhesive patch 4 and the primary TET module 6 in such a way, that the primary TET module 6 is located directly over the secondary TET module 16 located in the subcutaneous module 10 which has been fixed to the skull during the neurosurgery. In the alternative embodiment of the system according to the invention, it is possible to fix the primary TET module 6 by placing magnets in both the primary TET module 6 and the secondary TET module 13 and the subcutaneous module 10.

The location of the primary TET module 6 in relation to the subcutaneous module 10 is particularly crucial since the wireless transcutaneous transmission composed of two paths of transmission, i.e. data and energy, is optimized to send data and energy within a close range (around 2-3 cm), whereas the receiver and transmitter of energy and data should be parallel to each other (i.e. compatible with each other in terms of location).

Following the correct placement of the adhesive central adhesive patch 4a and the primary TET module 6 onto the scalp, other adhesive patches 4b with the insulated electrodes 3 are positioned on the head in such a way that they surround the central adhesive patch 4a and the primary TET module 6 and cover the greater portion of the scalp.

After all the adhesive patches 4b containing the insulated electrodes 3 are placed on the patient's head, the connecting wires 5 are connected to them to supply power from the primary TET module 6. Subsequently, the primary wire 8 is connected to the primary TET module 6 to transmit data and energy from an external control and power module 9.

In the present system, during the step of generation of alternating electric field, the distribution of the electric field at any given time is equal to the electric field of at least two pairs of electrodes, i.e. the insulated electrode 3 and the implantable insulated electrode 15 or of the insulated electrode 3 with the insulated electrode 3 or of the implantable insulated electrode 15 with the implantable insulated electrode 15 or of the insulated intracorporeal electrode 12 and the implantable insulated electrode 15 or the insulated intracorporeal electrode 12 with the insulated intracorporeal electrode 12. This means that at any given time there are at least two pairs of electrodes that are activated, which is pictured in FIG. 5A-F where four insulated electrodes 3 in the extracorporeal part 1 and four implantable insulated electrodes 15 are simultaneously active. FIG. 5A-5F present exemplary distributions of the electric field at a given time for four simultaneously active four pairs of electrodes, i.e. insulated electrodes and implantable insulated electrodes. Similar distributions of electric field may be achieved by use of intracorporeal insulated electrodes 12 instead of the extracorporeal insulated electrodes 3.

The system for treating tumors according to the invention, especially to treat intracranial tumors, can in the step when an alternating electric field is generated generate alternating electric field, preferably, continuously, of the frequency between 50 kHz and 500 kHz and/or the intensity of at least 0.5 V/cm, wherein the electric field is simultaneously generated in at least two directions.

In the alternative embodiment of the present system for treating tumors according to the invention, the number of electrodes 3, 12, 15 may be increased or decreased. Moreover, the number of implantable insulated electrodes 5 located in the implant 11 may differ from the number of insulated electrodes 3 located in the extracorporeal part 1.

FIG. 5A-5F presents the electric field distribution as a two-dimensional cross-section. The larger (external) square in each of the drawings depicts the insulated electrodes 3 in the extracorporeal part 1, whereas the smaller square depicts the implantable insulated electrodes 15. A direct or alternating current is supplied to the electrodes 3, 15. Appropriate connecting of given electrodes 3, 15 to the correct poles of at least one ultrasonic generator leads to the generation of a net electric field distribution at a given time. In this way, the distribution of the electric field may be manipulated in such a way that the resulting intensity of the field is greater in certain areas and smaller in other areas. FIG. 5A-F shows some of the numerous examples of electric field application.

The system for treating tumors, especially intracranial tumors, combining a technique of a continuous alternating electric field and ultrasounds powered by transcutaneous energy transmission operates in such way that it activates the intracranial module 11 (implant 11), which generates ultrasounds by the ultrasonic generator (16), several minutes before the planned administration of systemic chemotherapy. Such ultrasounds are generated by the ultrasonic generators 16 located inside the implant 11. The ultrasonic generators generate ultrasounds of the frequency around 200 kHz, but other frequencies can also be produced, e.g. of the frequency 100 kHz to 10 MHz, where they can be generated periodically or continuously. In one embodiment of the present invention, the ultrasonic generators 16 are piezoelectric crystals. Since the ultrasonic generators 16 are positioned in different planes, the ultrasonic wave project around the radius of the intracranial module 11, as illustrated in FIG. 7. This is fully intentional, as in the case of intracranial tumors treatment, the brain area where the blood-brain barrier should be disrupted for the chemotherapy is in direct proximity of the implant (11). At the same time, it is the area of the post-operative tumor bed. This means that it is highly probable that cancerous cells may be present in the post-operative bed.

The activation of the ultrasonic generators 16 occurs manually and wirelessly through the wireless communication module 39 which controls the device as it is operated by medical staff treating the individual in the hospital ward where the chemotherapy is administered or through other interface that the device may be equipped with, for example through a radio communication system or wired communication system, e.g. through a wired interface 40 (as shown in FIG. 6).

After connecting the external control panel to the external control and power module 9 (if it does not contain user interface) with the wireless communication module 39 and the command initiating the ultrasounds, such command, when wirelessly received e.g. through Bluetooth technology, is converted in the control and power module 9 to an optical signal and transmitted through an optical fiber in a cumulative conductor which is the primary wire 8, which further comprises the power supply line, and subsequently through the optical fiber to the optical communication module 7. Next, the optical communication module 7 located in the central adhesive patch 4a in the primary TET circuit 6, sends an optical signal to the analogous optical receiver module of the optical circuit 24 which is a component of the power supply path 10A of the subcutaneous module 10 that is placed under the skin. After the signal is received and processed, it is further transmitted through the secondary wire 14 which connects the implant 11 to the intracranial module 10. Then, the signal is conducted through the ultrasonic generators 16 which become activated.

Moreover, alternatively, the system can be automatically activated at a set hour. However, it should be emphasized that, in order to power the intracorporeal part 2 of the system, the user must wear the extracorporeal part 1 that transmits energy to the intracorporeal part 2. The implant 11 is powered by transcutaneous energy transmission (TET) that occurs through the inductive coupling of the primary winding of the transformer 29 in the extracorporeal part 1 with the secondary winding of the transformer 29 in the intracorporeal part 11, i.e. in the subcutaneous module 10 that is further wired to the implant 11.

It should also be emphasized, that in order to induce the therapeutic effect before or simultaneously with the activation of the ultrasonic generator 16, an intravascular contrast containing microbubbles must be administered—e.g. Optison™ (GE Healthcare, WI, USA), Definity® (Lantheus Medical Imaging, MA, USA) and SonoVue® (Bracco, Milan, Italy).

Shortly after administering the contrast containing microbubbles to the patient, it reaches the cerebral vessels and becomes subjected to ultrasounds generated by the ultrasonic generators 16. Subsequently, permeabilization takes place, i.e. the cell membrane of cerebral vessels undergoes an increase in permeability, and thus the blood-brain barrier becomes disrupted.

The anti-cancer action of the alternating electric field is enhanced by more effective application of chemotherapy, due to the intracranial generation of ultrasounds which temporarily disrupt, but do not cause damage to the blood-brain barrier and assure a much more efficient penetration of chemotherapeutic to the brain tissue. In turn, a much lower concentration of chemotherapeutic drugs may be administered while achieving the therapeutic concentration within the brain tissue and cancerous cells, which will contribute to a decrease in side effects of chemotherapy.

1. A system for treating tumors comprising an extracorporeal part (1) and an intracorporeal part (2), characterized in that:
the extracorporeal part (1) comprises a primary TET module (6) constituting a primary resonant circuit and an external control and power module (9) connected to the latter one, the intracorporeal part (2) comprising a subcutaneous module (10) connected to an implant (11);
wherein the subcutaneous module (10) comprising a secondary TET module (13) constituting a secondary resonant circuit that receives energy from the primary TET module (6), and the implant comprising at least two implanted insulated electrodes (15) and at least one ultrasonic generator (16).

2. The system according to claim 1, characterized in that the extracorporeal part (1) comprises at least two insulated electrodes (3) for generating an alternating electric field.

3. The system according to claim 1 or claim 2, characterized in that the subcutaneous module (10) consists of at least two insulated electrodes (3) for generating an alternating electric field.

4. The system according to claim 1 or claim 2 or claim 3, characterized in that the subcutaneous module (10) comprises an power supply path (10A) comprising at least the secondary TET module (13) the latter one comprising at least one secondary inductor (27A, 27B) of the secondary resonant circuit.

5. The system according to claim 4, characterized in that the subcutaneous module (10) further comprises a therapeutic path (10B) that comprises at least two subcutaneous insulated electrodes (12) arranged on a PCB (19) and a processor (31a).

6. The system according to claim 5, characterized in that the extracorporeal part (1) further comprises an optical communication module (7), and the subcutaneous module (10) comprises in the power supply path (10A)an optical receiver (24) that cooperates with the optical communication module (7) of the extracorporeal part (1).

7. The system according to claim 6, characterized in that the extracorporeal part (1) further comprises an optical communication module (7), and the optical receiver (24) that cooperates with the optical communication module (7) of the extracorporeal part (1) is located in the power supply path (10A) arranged in a subcutaneous power module (44).

8. The system according to any of claims 1 to 7, characterized in that the primary TET module (6) comprises a primary inductor (22), wherein the secondary TET module (13) comprising at least one secondary inductor (27A, 27B), the primary inductor (22) and at least one secondary inductor (27A, 27B) parts constituting a transformer (29).

9. The system according to any of claims 1 to 8, characterized in that the subcutaneous module (10) is wired to the implant (11), preferably through a secondary wire (14).

10. The system according to any of claims 1 to 9, characterized in that the extracorporeal part (1) further comprises an inverter (26) and a processor (31B) connected to: a sound communication circuit (32), an optical circuit (33), an input interface (37), a wireless communication module (39) connected to an antenna (38), a short-range wireless interface (41) and at least one multiplexer (36A);

11. The system according to claim 10, characterized in that at least one multiplexer (36A) is embedded in adhesive patches (4b) stuck on the head or is an integral part of the external control and power module (9).

12. The system according to any of claims 1 to 11, characterized in that if the extracorporeal part (1) comprises at least two insulated electrodes (3), the latter ones are embedded in the adhesive patches (4b) stuck on the head and are powered through connecting wires (5) connecting said at least two insulated electrodes (3) to the primary TET module (6) from which the primary wire (8) connecting the latter one to the external control and power module (9) is running.

13. The system according to any of claims 6, characterized in that the subcutaneous module (10) comprises further in the therapeutic path (10B) a rectifying circuit (34) and a smoothing circuit (35) connected to at least one secondary inductor (27A, 27B), and at least one multiplexer (36B), wherein the processor (31A) controls generation of ultrasounds from at least one ultrasonic generator (16) located in the implant (11), wherein preferably, at least one secondary inductor (27A, 27B), the optical receiver (24), the processor (31A), the rectifying circuit (34), the smoothing circuit (35) are located on the common PCB (19).

14. The system according to claim 13, characterized in that the processor (31A) mutually communicates with the processor (31B) by means of light impulses emitted through the skin by the optical communication module (7) that cooperates with the optical receiver (24) through means of light impulses emitted through the skin.

15. The system according to any of claims 1 to 14, characterized in that at least one ultrasonic generator (16) has the form of a piezoelectric crystal.

16. The system according to any of claims 1 to 15, characterized in that the external power source (9) constitutes a set of non-reusable batteries, an accumulator, an ‘energy harvesting’ generator which produces electric current from heat, light or movements, and/or a computer.

17. The system according to any of claims 1 to 16, characterized in that the intracorporeal part (2) is made of sterile, biocompatible material and/or materials which do not interfere with the MRI examination.

18. A method for treating tumors, especially intracranial tumors, characterized in that it comprises:
(a) a step of at least partial tumor resection;
(b) a step of locating the system according to any one of claims from 1 to 16 inside and on the body of a patient, wherein the implant 11 being placed in the tumor bed left after a fragmentary or total tumor resection;
(c) a step of generation of ultrasounds and/or a step of an alternating electric field generation prior to a step of administration of chemotherapy; and
(d) the step of administration of chemotherapy .

19. The method according to claim 18, characterized in that the distribution of the electric field during the step (c) at any given time is resultant of the electric field of at least two pairs of electrodes that generate electric field in different directions, preferably, of the insulated extracorporeal electrode (3) and the implantable insulated electrode (15) or of the insulated extracorporeal electrode (3) with the insulated electrode (3) or of the implantable insulated electrode (15) with the implantable insulated electrode (15) or of the insulated intracorporeal electrode (12) and the implantable insulated electrode (15) or of the insulated intracorporeal electrode (12) with the insulated intracorporeal electrode (12).

20. The method according to claim 18 or claim 19, characterized in that in the step (c) an alternating electric field is generated, preferably in a continuous manner, of the frequency between 50 kHz and 500 kHz and/or of the intensity of at least 0.5 V/cm, wherein the electric field being generated simultaneously in at least two directions.

21. The method according to claim 18 or claim 19 or claim 20, characterized in that in the step (c) the ultrasounds are generated, periodically or continuously, of the frequency between 100 kHz and 10 MHz.

22. A use according to any of claims 1 to 17 in treating tumors.

23. The use according to claim 22 in treating intracranial tumors.

24. The use according to claim 22 or claim 23 in treating intracranial tumors in combination with chemotherapy, wherein the system generates ultrasounds and/or alternating electric field prior to the step of administration of chemotherapy.
